(19) United States
(12) Patent Application Publication
(10) Pub. No.: US 2024/0282465 A1
(43) Pub. Date: Aug. 22, 2024

(54) METHOD OF PREDICTING INFECTIOUS DISEASE INFECTIONS AND SYSTEM THEREOF, DEVICE, AND STORAGE MEDIUM

(71) Applicant: ZHEJIANG LAB, Hangzhou (CN)

(72) Inventors: Zenghui XU, Hangzhou (CN); YU ZHANG, Hangzhou (CN); Zhan ZHANG, Hangzhou (CN); Ting YU, Hangzhou (CN); Jin ZHAO, Hangzhou (CN); Linlin HOU, Hangzhou (CN)

(21) Appl. No.: 18/212,727

(22) Filed: Jun. 22, 2023

(30) Foreign Application Priority Data
Feb. 17, 2023 (CN) 202310135076.4

(51) Int. Cl. G16H 50/80

(52) U.S. Cl. CPC G16H 50/80

(57) ABSTRACT

A method of predicting infectious disease infections and a system thereof, a device, and a storage medium are provided. An increment module controls an input module to obtain new incremental data in the current cycle in response to a preset instruction. A graph engine iteratively trains a first graph model based on new incremental data until the first graph model meets a convergence condition, so as to obtain a second graph model and perform dynamic updating of the graph model. Each region acts as a node in the graph model, each node feature is obtained based on the regional disease information, edges are defined by nodes connected to each other according to a geographic location relationship among regions, and each edge is assigned an edge weight according to regional population information. The updated graph model predicts infections based on data to be predicted that is selected by the interaction module.

### CROSS-REFERENCE TO RELATED APPLICATIONS

This application claims priority to Chinese patent application No. 202310135076.4, filed on Feb. 17, 2023, titled “METHOD OF PREDICTING INFECTIOUS DISEASE INFECTIONS AND SYSTEM THEREOF, DEVICE, AND STORAGE MEDIUM”, which is hereby incorporated by reference.

### TECHNICAL FIELD

The present disclosure relates to the field of predicting infections, and in particular, to a method of predicting infectious disease infections and a system thereof, a device, and a storage medium.

### BACKGROUND

Infectious diseases are usually spread by direct transfer of germs, viruses or other bacteria from one person to another, causing a very significant impact on a normal productive life of people. Prediction of future infections and prevalence of infectious diseases has been a major research subject in the field. Accurate prediction of the number of future infections may improve reference of measures against epidemics, which has great importance in a fight against infectious diseases.

In the related art, mathematical and statistical methods such as SIR (Susceptible-Infected-Removed) have been used for predicting infections, with a low level of intelligence and low prediction accuracy. Application of artificial intelligence techniques for predicting infectious disease infections can result in a high threshold of application due to complexity of technical solutions. Since infectious disease infections are dynamic over time, algorithms and systems have a dynamic requirement.

For the issue of low accuracy of predicting infectious disease infections in the related art, no effective solution is proposed.

### SUMMARY

A method of predicting infectious disease infections and a system thereof, a device, and a storage medium are provided in the present disclosure, to at least solve a problem of low accuracy of the prediction of infectious disease infection in the related art.

In a first aspect, the present disclosure provides a prediction method of infectious disease infection, applied in a prediction system of infectious disease infection which includes an increment module, an input module, a graph engine, and an interaction module.

The prediction method includes: the increment module controlling the input module to obtain new incremental data in the current cycle in response to a preset instruction; the graph engine iteratively training a first graph model based on the new incremental data until the first graph model meets a convergence condition, so as to obtain a second graph model; and the interaction module inputting data to be predicted to the second graph model to obtain a prediction result in response to a user instruction. The new incremental data includes regional disease information of at least one region in the current cycle. The first graph model is obtained by training based on historical data obtained in a previous cycle, each region acts as a node in the first graph model, each node feature is obtained based on the regional disease information, edges are defined by nodes connected to each other according to a geographic location relationship among regions, and each edge is assigned an edge weight according to regional population information. The prediction result includes the number of infected persons with infectious diseases in a target region at a future time.

In some embodiments, the regional disease information includes at least one of a regional population density, the number of regional susceptible persons, the number of regional infected persons, and the number of regional recovered persons.

In some embodiments, the first graph model includes a first node corresponding to a first region and a second node corresponding to a second region, and the regional disease information includes the number of persons moving from the first region to the second region, the number of persons moving from the second region to the first region, the number of persons moving from the first region to all other regions, and the number of persons moving from the second region to all other regions at a preset moment.

Each edge is assigned the edge weight according to regional population information by the following step: determining an edge weight from the first node to the second node according to a ratio between the number of persons moving from the first region to the second region and the number of persons moving from the first region to all other regions, a ratio between the number of persons moving from the second region to the first region and the number of persons moving from the second region to all other regions, and the total number of regions geographically adjacent to the first region.

In some embodiments, each edge is further assigned an edge weight according to regional population information by the following step: when the edge weight is less than a first threshold, rejecting the edge corresponding to the edge weight.

In some embodiments, the new incremental data includes regional disease information at a plurality of preset times, and the first graph model includes a long and short-term memory neural network.

The graph engine iteratively training the first graph model based on the new incremental data until the first graph model meets the convergence condition further includes: the graph engine sequentially inputting the new incremental data to the first graph model in accordance with the plurality of preset times, and performing forward prediction of the new incremental data via the long and short-term memory neural network to obtain a predicted number of infected persons; and obtaining a loss value based on the predicted number of infected persons and the true number of infected persons, and adjusting weight parameters of the first graph model based on the loss value.

In some embodiments, the first graph model further includes a fully connected layer, and the graph engine sequentially inputting the new incremental data to the first graph model in accordance with the plurality of preset times, and performing forward prediction of the new incremental data via the long and short-term memory neural network to obtain the predicted number of infected persons further includes: aggregating node information of adjacent nodes of a first node at the plurality of preset times to obtain aggregated data corresponding to the plurality of preset times; processing the aggregated data corresponding to the plurality of preset times by an activation function to obtain a plurality of embedded expression information; sequentially inputting the plurality of embedded expression information to the long and short-term memory neural network to obtain a hidden layer representation information; and inputting the hidden layer representation information to the fully connected layer to obtain the predicted number of infected persons. The node information includes node features of the adjacent nodes, and weight parameters corresponding to the first node and the adjacent nodes.

In a second aspect, the present disclosure provides a system of predicting infectious disease infections, including an increment module, an input module, a graph engine, and an interaction module.

The increment module is configured for controlling the input module to obtain new incremental data in the current cycle in response to a preset instruction, and the new incremental data includes regional disease information of at least one region in the current cycle.

The graph engine is configured for iteratively training a first graph model based on the new incremental data until the first graph model meets a convergence condition, so as to obtain a second graph model. The first graph model is obtained by training based on historical data obtained in a previous cycle, each region acts as a node in the first graph model, each node feature is obtained based on the regional disease information, nodes are connected to each other to form edges according to a geographic location relationship among regions, and each edge is assigned an edge weight according to regional population information.

The interaction module is configured for inputting data to be predicted to the second graph model to obtain a prediction result in response to a user instruction. The prediction result includes the number of infected persons with infectious diseases in a target region at a future time.

In some embodiments, the input module includes a crawler interface and a document interface. The crawler interface is configured for crawling regional disease information of the regions on the Internet. The document interface is configured for obtaining regional population information of the regions.

In some embodiments, the system of predicting infectious disease infections further includes a graph construction module, a graph database, or a display module.

The graph construction module is configured for generating graph data based on data obtained by the input module, and the graph data includes the node feature and the edge weight. The graph database is configured for storing the graph data. The display module is configured for receiving and displaying the prediction result.

In some embodiments, the user instruction to which the interaction module in response includes at least one of a control instruction for a startup time of the increment module, a hyperparameter setting instruction for the first graph model or the second graph model, and a scaling instruction for the display module.

In a third aspect, the present disclosure provides a computer device, including a processor and a memory that stores a computer program running on the processor. The computer program is executed by the processor to implement the steps of any one of the methods of predicting infectious disease infections in the first aspect.

In a fourth aspect, the present disclosure provides a storage medium having stored a computer program. The computer program is executed by a processor to implement the steps of any one of the methods of predicting infectious disease infections in the first aspect.

The details of one or more embodiments of the present disclosure are set forth in the accompanying drawings and the specification below. Other features, objects and advantages of the present disclosure will become apparent from the specification, drawings and claims.

### BRIEF DESCRIPTION OF THE DRAWINGS

In order to illustrate the embodiments of the present disclosure more clearly, the drawings used in the embodiments will be described briefly. Apparently, the following described drawings are merely for the embodiments of the present disclosure, and other drawings can be derived by one skilled in the art without any creative effort.

FIG. 1 is a block diagram of a hardware structure of a terminal for a method of predicting infectious disease infections in an embodiment of the present disclosure.

FIG. 2 is a flowchart diagram of a method of predicting infectious disease infections in an embodiment of the present disclosure.

FIG. 3 is a schematic diagram of a process for increment training of a graph model in an embodiment of the present disclosure.

FIG. 4 is a schematic diagram of a training process of a graph model in an embodiment of the present disclosure.

FIG. 5 is a schematic diagram of a system of predicting infectious disease infections in an embodiment of the present disclosure.

FIG. 6 is a schematic diagram of processing data for predicting infectious disease infections in an embodiment of the present disclosure.

### DETAILED DESCRIPTION

To make purposes, technical solutions and advantages of the present disclosure clearer, the present disclosure is described and explained below with reference to the accompanying drawings and embodiments of the present disclosure. It should be understood that the specific embodiments described herein are only used to interpret the present disclosure and are not intended to limit the present disclosure. Based on the embodiments provided in the present disclosure, all other embodiments obtained by one skilled in the art without performing creative labor fall within the scope of the present disclosure.

Obviously, the accompanying drawings in the following specification are only some examples or embodiments of the present disclosure, and the present disclosure may be applied to other similar scenarios in accordance with the accompanying drawings without creative labor to one skilled in the art. Furthermore, it is also understood that although the efforts made in development process may be complex and lengthy, for one skilled in the art related to the content disclosed in the present disclosure, some design, manufacturing or production variations based on the technical content disclosed in the present disclosure are only conventional technical means, and should not be understood as insufficient content disclosed in the present disclosure.

References to “embodiment” in the present application means that a particular feature, structure or property described in conjunction with the embodiment may be included in at least one embodiment of the present application. The occurrence of the phrase in various positions of the specification does not necessarily refer to the same embodiment, nor is it a separate or alternative embodiment that is mutually exclusive with other embodiments. One skilled in the art expressly and implicitly may understand that the embodiments described in the present application may be combined with other embodiments without conflict.

Unless otherwise defined, all technical and scientific terms used herein have the same meaning as one skilled in the art would understand. The term “one”, “a”, “an”, “the” and other similar words as used in the present disclosure do not indicate quantitative limitations, and they can be singular or plural. The term “plurality” in the present disclosure refers to two or more. The terms “include”, “comprise”, “have”, and any variation thereof, as used in the present disclosure, are intended to cover a non-exclusive inclusion. For example, a process, a method, a system, a product, or a device including a series of steps or modules (units) is not limited to the listed steps or units, but may also include steps or units that are not listed, or may also include other steps or units that are inherent to the process, the method, the product, or the device. The term “plurality” as used in the present disclosure refers to two or more.

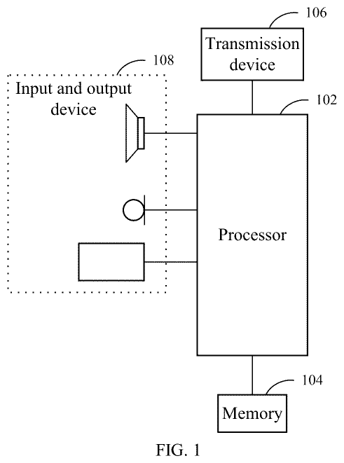

A method embodiment provided in the embodiment of the present disclosure may be performed in a terminal, a computer, or similar computing device. For example, running on a terminal, FIG. 1 is a block diagram of a hardware structure of a terminal for a method of predicting infectious disease infections in an embodiment of the present disclosure. Referring to FIG. 1, the terminal may include one or more processors 102 (only one is shown in FIG. 1) and a memory 104 configured for storing data. The processor 102 may include, but not limited to, processing devices such as a Microcontroller Unit (MCU) or a Field Programmable Gate Array (FPGA). The terminal may also include a transmission device 106 for communication functions and an input and output device 108. It could be understood by one skilled in the art that the structure shown in FIG. 1 is only schematic and that it does not limit the structure of the above terminal. For example, the terminal may also include more or fewer components than shown in FIG. 1, or have a different configuration than that shown in FIG. 1.

The memory 104 may be configured to store computer programs, for example, software programs and modules of application software, such as a computer program corresponding to a method of predicting infectious disease infections in the present disclosure. The processor 102 may perform various functional applications as well as data processing, i.e., implement the method described above, by performing the computer program stored in the memory 104. The memory 104 may include a high-speed random memory, and may also include a non-volatile memory such as one or more magnetic storage devices, flash memories, or other non-volatile solid-state memories. In some embodiments, the memory 104 may further include a memory that is remotely located relative to the processor 102, and the remote memory may be connected to the terminal via a network. The network may include, but is not limited to, the Internet, a corporate intranet, a local area network, a mobile communication network, and combinations thereof.

The transmission device 106 is configured to receive or send data via a network. The network may include a wireless network provided by a communication provider of the terminal. In an embodiment, the transmission device 106 may include a Network Interface Controller (NIC) that can be connected to other network devices via a base station so that the transmission device 106 can communicate with the Internet. In an embodiment, the transmission device 106 may be a Radio Frequency (RF) module configured to communicate with the Internet wirelessly.

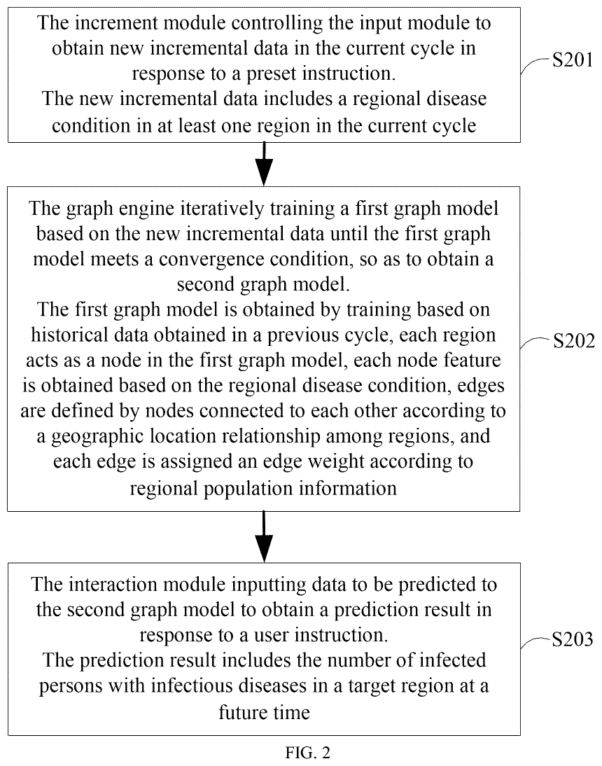

In an embodiment, a method of predicting infectious disease infections is provided. The prediction method is applied in a system of predicting infectious disease infections which includes an increment module, an input module, a graph engine, and an interaction module. FIG. 2 is a flowchart diagram of a method of predicting infectious disease infections in an embodiment of the present disclosure. Referring to FIG. 2, the flowchart includes the following steps.

Step 201 includes the increment module controlling the input module to obtain new incremental data in the current cycle in response to a preset instruction. The new incremental data includes regional disease information of at least one region in the current cycle.

The preset instruction may refer to a startup instruction of the increment module which is input by the user. The input module may crawl infectious status data of each region, including the number of susceptible persons, the number of infected persons, and the number of recovered persons in each region in the current cycle. The increment module may control the input module to re-crawl and input the latest data at regular intervals. Based on the new incremental data, the increment module may invoke the graph engine for increment training to achieve update of the graph model.

Step 202 includes the graph engine iteratively training a first graph model based on the new incremental data until the first graph model meets a convergence condition, so as to obtain a second graph model. The first graph model is obtained by training based on historical data obtained in a previous cycle, each region acts as a node in the first graph model, each node feature is obtained based on the regional disease information, edges are defined by nodes connected to each other according to a geographic location relationship among regions, and each edge is assigned an edge weight according to regional population information. The regional population information may include population migration data among the regions, such as the number of persons moving from one region to another region in the current cycle.

The first graph model may be a graph model obtained by training based on historical infection data, and the second graph model may be an updated graph model obtained by increment training based on new incremental infection data. Each region may act as a node, and when two regions are geographically adjacent to each other, an edge may be defined between the nodes corresponding to the two regions. The edge weight may represent a weight of the node in one region pointing to the node in another region at a certain moment, determined by both geographic locations of the regions and the population migration data between the regions.

Step 203 includes the interaction module inputting data to be predicted to the second graph model to obtain a prediction result in response to a user instruction. The prediction result includes the number of infected persons with infectious diseases in a target region at a future time.

The user instruction may refer to an instruction to select data to be predicted as needed, and an instruction to set parameters of the first graph model and the second graph model. After defining parameters and inputting the new incremental data, the first graph model may perform model training for forward propagation to obtain the second graph model, and the second graph model may perform prediction calculations to predict future infectious disease infections and present the prediction result.

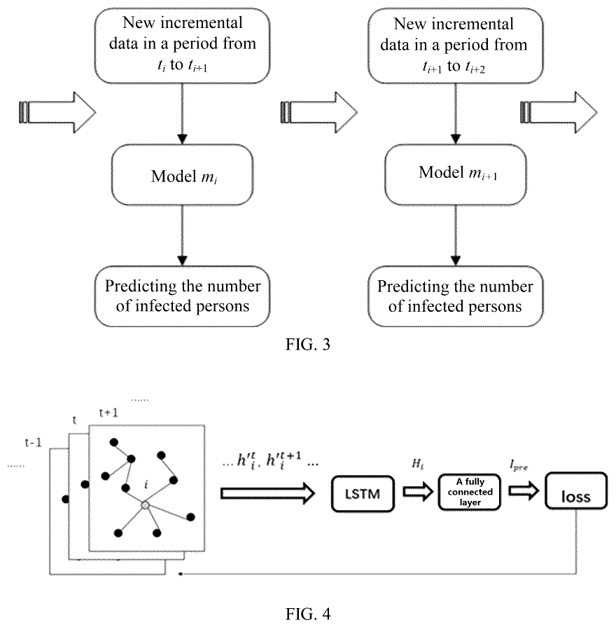

FIG. 3 is a schematic diagram of a process for increment training of a graph model in an embodiment of the present disclosure. Referring to FIG. 3, a model mᵢ corresponds to the first graph model, and a model mᵢ₊₁ corresponds to the second graph model. The first graph model mᵢ may be obtained by training based on past data in a period from tᵢ to tᵢ₊₁, and the second graph model mᵢ₊₁ may be obtained by training based on new crawled data in a period from tᵢ₊₁ to tᵢ₊₂. The graph model may be updated continuously based on incremental data via increment training to achieve dynamic prediction of infectious disease infections.

At the step 201 to step 203, the increment module controls the input module to obtain new incremental data in the current cycle in response to a preset instruction. The graph engine iteratively trains a first graph model based on the new incremental data until the first graph model meets a convergence condition, so as to obtain a second graph model and perform dynamic updating of the graph model. Each region acts as a node in the graph model, each node feature is obtained based on the regional disease information, edges are defined by nodes connected to each other according to a geographic location relationship among regions, and each edge is assigned an edge weight according to regional population information. The updated graph model predicts infectious disease infections based on data to be predicted that is selected by the interaction module. The above solution may solve a problem of low accuracy of predicting of infectious disease infections in the related art, and have an advantage of improving accuracy of predicting infectious disease infections.

In some embodiments, the regional disease information may include at least one of a regional population density, the number of regional susceptible persons, the number of regional infected persons, and the number of regional recovered persons.

A node feature of a region denoted as Nᵢ^{t} at a time denoted as t may be aggregated directly according to the regional disease information. A specific calculation formula may be:

$${N}_{i}^{t} = [ {P}_{i}^{t} \vert \vert {N}_{i}^{t} \vert \vert {I}_{i}^{t} \vert \vert {R}_{i}^{t} ]$$

i represents a first node, j represents a second node, Pᵢ^{t} represents a population density of a first region corresponding to node i at the time t, Sᵢ^{t} represents the number of susceptible persons of the first region at the time t, Iᵢ^{t} represents the number of infected persons of the first region at the time t, and Rᵢ^{t} represents the number of recovered persons of the first region at the time t.

In some embodiments, the first graph model may include the first node corresponding to the first region and the second node corresponding to a second region, and the regional disease information may include the number of persons moving from the first region to the second region, the number of persons moving from the second region to the first region, the number of persons moving from the first region to all other regions, and the number of persons moving from the second region to all other regions at a preset moment. Each edge may be assigned the edge weight according to the regional population information by the following step: determining an edge weight from the first node to the second node according to a ratio between the number of persons moving from the first region to the second region and the number of persons moving from the first region to all other regions, a ratio between the number of persons moving from the second region to the first region and the number of persons moving from the second region to all other regions, and the total number of regions geographically adjacent to the first region.

The edge weight may indicate strength of an interrelationship between the first node and the second node. When determining the edge weight from the first node to the second node, it is also necessary to set hyperparameters of the first node and the second node.

A specific calculation formula of the edge weight from the first node to the second node may be:

$${e}_{ij}^{t} = α Ne ( ij ) + β ( \frac{{Re}_{ij}^{t}}{{Re}_{i}^{t}} + \frac{{Re}_{ji}^{t}}{{Re}_{j}^{t}} )$$

$$Ne ( ij ) = \{ \begin{matrix}1 / L ( i ) , & i is geographically adjacent to j \\ 0 , & i is not geographically adjacent to j\end{matrix}$$

L(i) represents the total number of regions geographically adjacent to the first region, Reᵢⱼ^{t} represents the number of persons moving from the first region to the second region at the time t, Reⱼᵢ^{t} represents the number of persons moving from the second region to the first region at the time t, Reᵢ^{t} represents the number of persons moving from the first region to all other regions at the time t, Reⱼ^{t} represents the number of persons moving from the second region to all other regions at the time t, and α and β represent the hyperparameters of the first node and the second node.

In some embodiments, each edge may be further assigned the edge weight according to the regional population information by the following step: when the edge weight is less than a first threshold, rejecting the edge corresponding to the edge weight.

When the edge weight eᵢⱼ^{t} is less than 0.2, the edge corresponding to the edge weight may be rejected to prevent over-connecting in the graph model.

In some embodiments, the new incremental data may include regional disease information at a plurality of preset times, and the first graph model may include a long and short-term memory neural network.

The graph engine iteratively training the first graph model based on the new incremental data until the first graph model meets the convergence condition may further include: the graph engine sequentially inputting the new incremental data to the first graph model in accordance with the plurality of preset times, and performing forward prediction of the new incremental data via the long and short-term memory neural network to obtain a predicted number of infected persons; and obtaining a loss value based on the predicted number of infected persons and the true number of infected persons, and adjusting weight parameters of the first graph model based on the loss value.

The loss value predicted each time may be fed back to the graph model, which may adjust composition and parameters according to the loss value. The loss value may be a mean squared difference between the true number of infected persons and the predicted number of infected persons. A special calculation formula may be: Loss=MSE (Iₚᵣₑ, Iₜᵣᵤ). Loss represents the loss value, MSE represents the mean squared difference, Iₚᵣₑ represents the predicted number of infected persons, Iₜᵣᵤ represents the true number of infected persons.

In some embodiments, FIG. 4 is a schematic diagram of a training process of a graph model in an embodiment of the present disclosure. Referring to FIG. 4, the first graph model may further include a fully connected layer. The graph engine sequentially inputting the new incremental data to the first graph model in accordance with the plurality of preset times, and performing forward prediction of the new incremental data via the long and short-term memory neural network to obtain the predicted number of infected persons may further include: aggregating node information of adjacent nodes of a first node at the plurality of preset times to obtain aggregated data corresponding to the plurality of preset times; processing the aggregated data corresponding to the plurality of preset times by an activation function to obtain a plurality of embedded expression information; sequentially inputting the plurality of embedded expression information to the long and short-term memory neural network to obtain a hidden layer representation information; and inputting the hidden layer representation information to the fully connected layer to obtain the predicted number of infected persons.

The first node i may aggregate node information of adjacent nodes of the first node via an aggregation function, which introduces nonlinear features to the neural network and enhances expression capability of the neural network. A special calculation formula may be: h′ᵢ^{t}=σ(Σ_{j∈N}(i)wᵢⱼNⱼ^{t}). h′ᵢ^{t} represents the embedded expression information of the first node i, σ represents the activation function, N(i) represents an edge of the first node i, wᵢⱼ represents the weight parameters of the first node i and the adjacent nodes j of the first node. The weight parameters may be setting parameters of the neural network to control segmentation of views in the graph model, and may be obtained by training. The plurality of embedded expression information may be sequentially input to the long and short-term memory neural network to obtain the hidden layer representation information denoted as Hᵢ. The hidden layer representation information may be input to the fully connected layer to perform prediction to obtain the predicted number of infected persons.

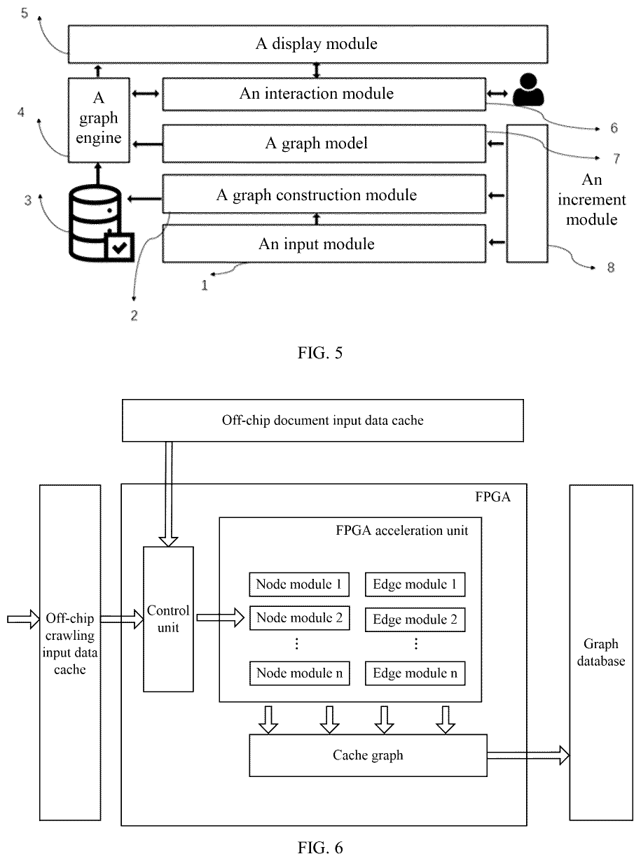

In some embodiments, the present disclosure further provides a system of predicting infectious disease infections. Referring to FIG. 5, the system of predicting infectious disease infections includes an increment module 8, an input module 1, a graph engine 4, and an interaction module 6. The increment module 8 is configured for controlling the input module 1 to obtain new incremental data in the current cycle in response to a preset instruction, and the new incremental data includes regional disease information of at least one region in the current cycle. The graph engine 4 is configured for iteratively training a first graph model based on the new incremental data until the first graph model meets a convergence condition, so as to obtain a second graph model. The first graph model is obtained by training based on historical data obtained in a previous cycle, each region acts as a node in the first graph model, each node feature is obtained based on the regional disease information, nodes are connected to each other to form edges according to a geographic location relationship among regions, and each edge is assigned an edge weight according to regional population information. The interaction module 6 is configured for inputting data to be predicted to the second graph model to obtain a prediction result in response to a user instruction. The prediction result includes the number of infected persons with infectious diseases in a target region at a future time.

In some embodiments, the input module 1 may include a crawler interface and a document interface. The crawler interface is configured for crawling regional disease information of the regions on the Internet. The document interface is configured for obtaining regional population information of the regions.

The crawler interface may crawl the daily number of infected persons the daily number of recovered persons in the regions on the Internet. A uniform resource locator (URL) of the crawler interface may be generally data in an official public URL of a target infectious disease, and a system of the present disclosure may support template file input in prescribed formats such as txt, csv, xls, etc. The document interface may input a geographic location of each region, a total population of each region, a population density of each region, and a population migration data between the regions.

In some embodiments, the system of predicting infectious disease infections may further include a graph construction module 2, a graph database 3, or a display module 5. The graph construction module 2 is configured for generating graph data based on data obtained by the input module, and the graph data may include the node feature and the edge weight. The graph database 3 is configured for storing the graph data. The display module 5 is configured for receiving and displaying the prediction result.

Referring to FIG. 6, document data and crawler data obtained by the input module 1 are saved in a cache without additional reading or storage, and then directly input to the FPGA for calculation. Calculation results are saved to the graph database 3. In the process of performing edge calculation, each region is assigned to an edge calculation module in the FPGA, and the calculation is performed in parallel using the population migration and geographic data in the document data. In the process of performing node calculation, the crawler interface may immediately input data of each region into a node calculation module in the FPGA for performing node feature calculation in real time in combination with the document data. By avoiding additional cache storage and reading, the parallel performing of multiple node calculation modules and edge calculation modules may greatly improve speed and efficiency between data input and graph construction.

In some embodiments, the user instruction to which the interaction module 6 in response may include at least one of a control instruction for a startup time of the increment module 8, a hyperparameter setting instruction for the first graph model or the second graph model, and a scaling instruction for the display module 5.

The interaction module 6 may set a startup time of the increment module 8 to control dynamic real-time performance of the prediction system. The interaction module 6 may also set a name of the graph database 3 and the hyperparameters of the graph model 7 to realize overall control of the prediction system. The interaction module 6 may also set a size and a scaling ratio of drawings of the display module 5 to optimize visualization performance of the display module 5.

Referring to FIG. 5, an overall operation process of the system of predicting infectious disease infections may be as follows. The input module 1 may input data to the graph construction module 2, the graph construction module 2 may generate the graph data, and the graph database 3 may store the graph data generated by the graph construction module 2. The interaction module 6 may give instructions for selecting graph data and setting parameters of the graph model as input by the user, control the graph engine 4 to train the model for prediction, and send the prediction result to the display module 5 for display. When the data is updated, the increment module may control the input module 1 to input the new incremental data to the graph construction module 2 for increment construction, and call the graph engine 4 for increment training to obtain the updated second graph model.

The present disclosure further provides a computer device, including a processor and a memory that stores a computer program running on the processor. The computer program is executed by the processor to implement the steps of any one of the methods of predicting infectious disease infections in the above embodiments.

The present disclosure further provides a storage medium having stored a computer program. The computer program is executed by a processor to implement the steps of any one of the methods of predicting infectious disease infections in the above embodiments.

One skilled in the art can understand that implementing all or part of the processes in the methods of the above embodiments may be accomplished by directing the associated hardware by means of a computer program, which may be stored in a non-volatile computer readable storage medium. When the computer program is executed, processes of the above methods in the embodiments may be included. Any reference to a memory, a storage, a database, or other media used in the embodiments provided in the present disclosure may include either or both of a non-volatile memory and a volatile memory. The non-volatile memory may include a read-only memory (ROM), a programmable ROM (PROM), an electrically programmable ROM (EPROM), an electrically erasable programmable ROM (EEPROM), or a flash memory. The volatile memory may include a random-access memory (RAM) or an external cache memory. By way of illustration and not limitation, the RAM is available in a variety of forms, such as a static RAM (SRAM), a dynamic RAM (DRAM), a synchronous DRAM (SDRAM), a double data rate SDRAM (DDRSDRAM), an enhanced SDRAM (ESDRAM), a synchronous link DRAM (SLDRAM), a Rambus DRAM (RDRAM), a direct Rambus DRAM (DRDRAM), etc.

The technical features of the above-described embodiments may be combined in any combination. For the sake of brevity of specification, not all possible combinations of the technical features in the above embodiments are described. However, as long as there is no contradiction between the combinations of these technical features, all should be considered as within the scope of this disclosure.

The above-described embodiments are merely illustrative of several embodiments of the present disclosure, and the specification thereof is relatively specific and detailed, but is not to be construed as limiting the scope of the disclosure. It should be noted that a number of variations and modifications may be made by one skilled in the art without departing from the spirit and scope of the disclosure. Therefore, the scope of the disclosure should be determined by the appended claims.

What is claimed is:

1. A method of predicting infectious disease infections, applied in a system of predicting infectious disease infections which comprises an increment module, an input module, a graph engine, and an interaction module, wherein the prediction method comprises:
the increment module controlling the input module to obtain new incremental data in the current cycle in response to a preset instruction, wherein the new incremental data comprises regional disease information of at least one region in the current cycle;
the graph engine iteratively training a first graph model based on the new incremental data until the first graph model meets a convergence condition, so as to obtain a second graph model, wherein the first graph model is obtained by training based on historical data obtained in a previous cycle, each region acts as a node in the first graph model, each node feature is obtained based on the regional disease information, edges are defined by nodes connected to each other according to a geographic location relationship among regions, and each edge is assigned an edge weight according to regional population information; and
the interaction module inputting data to be predicted to the second graph model to obtain a prediction result in response to a user instruction, wherein the prediction result comprises the number of infected persons with infectious diseases in a target region at a future time.

2. The method of predicting infectious disease infections of claim 1, wherein the regional disease information comprises at least one of a regional population density, the number of regional susceptible persons, the number of regional infected persons, and the number of regional recovered persons.

3. The method of predicting infectious disease infections of claim 2, wherein the first graph model comprises a first node corresponding to a first region and a second node corresponding to a second region, the regional disease information comprises the number of persons moving from the first region to the second region, the number of persons moving from the second region to the first region, the number of persons moving from the first region to all other regions, and the number of persons moving from the second region to all other regions at a preset moment; and each edge is assigned the edge weight according to regional population information by the following step:
determining an edge weight from the first node to the second node according to a ratio between the number of persons moving from the first region to the second region and the number of persons moving from the first region to all other regions, a ratio between the number of persons moving from the second region to the first region and the number of persons moving from the second region to all other regions, and the total number of regions geographically adjacent to the first region.

4. The method of predicting infectious disease infections of claim 3, wherein each edge is further assigned an edge weight according to regional population information by the following step:
when the edge weight is less than a first threshold, rejecting the edge corresponding to the edge weight.

5. The method of infectious disease infections of claim 1, wherein the new incremental data comprises regional disease information at a plurality of preset times, the first graph model comprises a long and short-term memory neural network, and the graph engine iteratively training the first graph model based on the new incremental data until the first graph model meets the convergence condition further comprises:
the graph engine sequentially inputting the new incremental data to the first graph model in accordance with the plurality of preset times, and performing forward prediction of the new incremental data via the long and short-term memory neural network to obtain a predicted number of infected persons; and
obtaining a loss value based on the predicted number of infected persons and the true number of infected persons, and adjusting weight parameters of the first graph model based on the loss value.

6. The method of predicting infectious disease infections of claim 5, wherein the first graph model further comprises a fully connected layer, and the graph engine sequentially inputting the new incremental data to the first graph model in accordance with the plurality of preset times, and performing forward prediction of the new incremental data via the long and short-term memory neural network to obtain the predicted number of infected persons further comprises:
aggregating node information of adjacent nodes of a first node at the plurality of preset times to obtain aggregated data corresponding to the plurality of preset times, wherein the node information comprises node features of the adjacent nodes, and weight parameters corresponding to the first node and the adjacent nodes;
processing the aggregated data corresponding to the plurality of preset times by an activation function to obtain a plurality of embedded expression information;
sequentially inputting the plurality of embedded expression information to the long and short-term memory neural network to obtain a hidden layer representation information; and
inputting the hidden layer representation information to the fully connected layer to obtain the predicted number of infected persons.

7. A system of predicting infectious disease infections, comprising an increment module, an input module, a graph engine, and an interaction module,
wherein the increment module is configured for controlling the input module to obtain new incremental data in the current cycle in response to a preset instruction, wherein the new incremental data comprises regional disease information of at least one region in the current cycle;
the graph engine is configured for iteratively training a first graph model based on the new incremental data until the first graph model meets a convergence condition, so as to obtain a second graph model, wherein the first graph model is obtained by training based on historical data obtained in a previous cycle, each region acts as a node in the first graph model, each node feature is obtained based on the regional disease information, nodes are connected to each other to form edges according to a geographic location relationship among regions, and each edge is assigned an edge weight according to regional population information; and
the interaction module is configured for inputting data to be predicted to the second graph model to obtain a prediction result in response to a user instruction, wherein the prediction result comprises the number of infected persons with infectious diseases in a target region at a future time.

8. The system of predicting infectious disease infections of claim 7, wherein the input module comprises a crawler interface and a document interface;
the crawler interface is configured for crawling regional disease information of the regions on the Internet; and
the document interface is configured for obtaining regional population information of the regions.

9. The system of predicting infectious disease infections of claim 7, further comprising a graph construction module, a graph database, or a display module;
wherein the graph construction module is configured for generating graph data based on data obtained by the input module, wherein the graph data comprises the node feature and the edge weight;
the graph database is configured for storing the graph data; or
the display module is configured for receiving and displaying the prediction result.

10. The system of predicting infectious disease infections of claim 7, wherein the user instruction to which the interaction module in response comprises at least one of a control instruction for a startup time of the increment module, a hyperparameter setting instruction for the first graph model or the second graph model, and a scaling instruction for the display module.

11. A computer device, comprising a processor and a memory that stores a computer program running on the processor, wherein the computer program is executed by the processor to implement the steps of the method of predicting infectious disease infections of claim 1.

12. The computer device of claim 11, wherein the regional disease information comprises at least one of a regional population density, the number of regional susceptible persons, the number of regional infected persons, and the number of regional recovered persons.

13. The computer device of claim 12, wherein the first graph model comprises a first node corresponding to a first region and a second node corresponding to a second region, the regional disease information comprises the number of persons moving from the first region to the second region, the number of persons moving from the second region to the first region, the number of persons moving from the first region to all other regions, and the number of persons moving from the second region to all other regions at a preset moment; and each edge is assigned the edge weight according to regional population information by the following step:
determining an edge weight from the first node to the second node according to a ratio between the number of persons moving from the first region to the second region and the number of persons moving from the first region to all other regions, a ratio between the number of persons moving from the second region to the first region and the number of persons moving from the second region to all other regions, and the total number of regions geographically adjacent to the first region.

14. The computer device of claim 13, wherein each edge is further assigned an edge weight according to regional population information by the following step:
when the edge weight is less than a first threshold, rejecting the edge corresponding to the edge weight.

15. The computer device of claim 11, wherein the new incremental data comprises regional disease information at a plurality of preset times, the first graph model comprises a long and short-term memory neural network, and the graph engine iteratively training the first graph model based on the new incremental data until the first graph model meets the convergence condition further comprises:
the graph engine sequentially inputting the new incremental data to the first graph model in accordance with the plurality of preset times, and performing forward prediction of the new incremental data via the long and short-term memory neural network to obtain a predicted number of infected persons; and
obtaining a loss value based on the predicted number of infected persons and the true number of infected persons, and adjusting weight parameters of the first graph model based on the loss value.

16. The computer device of claim 15, wherein the first graph model further comprises a fully connected layer, and the graph engine sequentially inputting the new incremental data to the first graph model in accordance with the plurality of preset times, and performing forward prediction of the new incremental data via the long and short-term memory neural network to obtain the predicted number of infected persons further comprises:
aggregating node information of adjacent nodes of a first node at the plurality of preset times to obtain aggregated data corresponding to the plurality of preset times, wherein the node information comprises node features of the adjacent nodes, and weight parameters corresponding to the first node and the adjacent nodes;
processing the aggregated data corresponding to the plurality of preset times by an activation function to obtain a plurality of embedded expression information;
sequentially inputting the plurality of embedded expression information to the long and short-term memory neural network to obtain a hidden layer representation information; and
inputting the hidden layer representation information to the fully connected layer to obtain the predicted number of infected persons.

17. A storage medium having stored a computer program, wherein the computer program is executed by a processor to implement the steps of the method of predicting infectious disease infections of claim 1.

18. The storage medium of claim 17, wherein the regional disease information comprises at least one of a regional population density, the number of regional susceptible persons, the number of regional infected persons, and the number of regional recovered persons.

19. The storage medium of claim 18, wherein the first graph model comprises a first node corresponding to a first region and a second node corresponding to a second region, the regional disease information comprises the number of persons moving from the first region to the second region, the number of persons moving from the second region to the first region, the number of persons moving from the first region to all other regions, and the number of persons moving from the second region to all other regions at a preset moment; and each edge is assigned the edge weight according to regional population information by the following step:
determining an edge weight from the first node to the second node according to a ratio between the number of persons moving from the first region to the second region and the number of persons moving from the first region to all other regions, a ratio between the number of persons moving from the second region to the first region and the number of persons moving from the second region to all other regions, and the total number of regions geographically adjacent to the first region.

20. The storage medium of claim 19, wherein each edge is further assigned an edge weight according to regional population information by the following step:
when the edge weight is less than a first threshold, rejecting the edge corresponding to the edge weight.
